(19) United States
(12) Patent Application Publication
(10) Pub. No.: US 2010/0080765 A1
(43) Pub. Date: Apr. 1, 2010

(54) EYELASH MAKEUP COMPOSITION AND CONDITIONING KIT

(75) Inventors: Christophe DUMOUSSEAUX, Antony (FR); Carole Le Merrer, Montrouge (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: L'OREAL, Paris (FR)

(21) Appl. No.: 12/568,023

(22) Filed: Sep. 28, 2009

Related U.S. Application Data
(60) Provisional application No. 61/104,299, filed on Oct. 10, 2008.

(30) Foreign Application Priority Data
Sep. 30, 2008 (FR) 08 56544

(51) Int. Cl. A61K 8/72; A61Q 1/10; A61Q 5/00

(52) U.S. Cl. 424/70.7; 424/70.11

(57) ABSTRACT

A composition for making up or caring for keratin fibres, containing a continuous oily phase, at least one compound chosen from copolymers of an alkene and of vinyl acetate, and at least one polar wax.

### REFERENCE TO PRIOR APPLICATIONS

This application claims priority to U.S. provisional application Ser. No. 61/104,299, filed Oct. 10, 2008; and to French patent application 08 56544, filed Sep. 30, 2008, both incorporated herein by reference.

### FIELD OF THE INVENTION

The present invention relates to a composition, preferably a non-therapeutic cosmetic composition, preferably for making up or caring for keratin fibres (such as the eyelashes, the eyebrows or the hair), comprising a continuous oily phase, at least one compound chosen from copolymers of an alkene and of vinyl acetate and at least one polar wax.

The invention is preferably in the form of a mascara or an eyebrow product. More especially, the invention relates to a mascara.

### BACKGROUND OF THE INVENTION

The term “mascara” means a composition intended to be applied to keratin fibres: it may be a keratin fibre makeup composition, a keratin fibre makeup base, or base coat, a composition to be applied over a mascara, also known as a top coat, or a cosmetic composition for treating keratin fibres. The mascara is more particularly intended for human keratin fibres, but also for false eyelashes.

Eye makeup compositions, also known as “mascaras” for the eyelashes, generally consist of a wax or a mixture of waxes dispersed using at least one surfactant in an aqueous phase also containing polymers and pigments.

It is generally by means of the qualitative and quantitative choice of the waxes and polymers that the desired application specificities for makeup compositions are adjusted, for instance their fluidity, their covering power and/or their curling power. Thus, it is possible to produce various compositions, which, when applied especially to the eyelashes, induce a variety of effects such as lengthening, curling and/or thickening (charging effect).

The present invention is more particularly directed towards providing a composition that is useful for producing a heavy makeup result on keratin fibres and especially the eyelashes, which is also known as charging makeup. For the purposes of the present invention, the term “keratin fibres” covers the hair, the eyelashes and the eyebrows and also extends to synthetic wigs and false eyelashes.

With the makeup compositions that are currently available, this effect is generally obtained by superimposing several coats of the makeup composition onto the keratin fibres and more particularly the eyelashes. Moreover, in the particular case of the eyelashes, this effect is very often associated with an aggregation of several eyelashes together, i.e. a non-individualization of the eyelashes.

To do this, certain makeup compositions have been proposed, which have a concentration of solids sufficient to significantly charge the eyelashes from their very first contact with the compositions. However, such compositions may become too thick on application and may no longer have the deformability necessary to enable them to be applied uniformly over the entire surface of the eyelashes.

In addition, such compositions are not ideal in terms of pleasantness and comfort on application: they occasionally have a dragging and dry aspect during application.

For obvious reasons, it would be advantageous to obtain a composition that simultaneously offers an immediate charging effect, sufficient consistency and good slipperiness thus without a dragging or dry sensation when applied, especially with a brush.

The inventors have discovered that the properties described above are obtained by using a composition comprising a continuous oily phase, at least one compound chosen from copolymers of an alkene and of vinyl acetate and at least one polar wax.

### DETAILED DESCRIPTION OF THE PREFERRED EMBODIMENTS

More specifically, one subject of the invention is a composition, preferably suitable for making up or caring for keratin fibres, comprising a continuous oily phase, at least one compound chosen from copolymers of an alkene and of vinyl acetate, and at least one polar wax.

A subject of the present invention is also a non-therapeutic cosmetic process for making up or caring for keratin fibres, comprising the application to the keratin fibres of a composition according to the invention.

A subject of the present invention is also a kit for conditioning a composition for making up and/or caring for keratin fibres, especially the eyelashes or the eyebrows, comprising:

i) a container delimiting at least one compartment, and

ii) a composition according to the invention placed inside the compartment.

The composition preferably comprises a physiologically acceptable medium, i.e. a non-toxic medium that may be applied to keratin fibres, such as human eyelashes, eyebrows or hair, and that is especially compatible with the area of the eyes.

Copolymers of an Alkene and of Vinyl Acetate, in Particular Ethylene-Based Copolymers

The term “alkene” means a hydrocarbon monomer whose linear or branched carbon-based chain contains a carbon-carbon double bond. The alkene according to the invention thus lacks heteroatoms.

The term “copolymer” means a polymer comprising two different monomers.

Such copolymers have the advantage of combining a low melting point, good compatibility with hydrocarbon-based waxes and good adhesion to various supports, especially keratin fibres.

The following copolymers may be used: vinyl acetate/octadecene or vinyl acetate/1-dodecene, preferably copolymers of ethylene and of vinyl acetate.

Copolymers of ethylene and of vinyl acetate preferably comprising between 5% and 50% by weight, preferably between 10% and 45% by weight and preferably between 20% and 40% by weight of vinyl acetate relative to the total weight of the polymer are preferably used. Increasing the content of vinyl acetate monomer in the copolymer makes it possible to increase the adhesion to keratin fibres.

Examples of ethylene/vinyl acetate copolymers that may be mentioned include those sold under the name Elvax by the company Du Pont de Nemours and in particular the compounds Elvax 40W, Elvax 140W, Elvax 200W, Elvax 205W, Elvax 210W and Elvax 310.

The products sold under the name Evatane by the company Arkema, such as Evatane 28-800, may also be mentioned.

These copolymers make it possible to thicken the fatty phase, in particular to thicken the waxy mixture at a temperature above the melting point of the waxes. This high viscosity also makes it possible to achieve very good dispersion of the pigments at elevated temperature in the fatty phase and to obtain a very good intensity of colour of the formulation.

To achieve efficient thickening of the fatty phase, these copolymers preferably have a weight-average molecular mass Mw, expressed as polystyrene equivalents, of greater than 30 000 daltons, preferably greater than 50 000 daltons and even more preferentially greater than 60 000 daltons.

The weight-average molecular mass Mw is advantageously chosen between 30 000 and 300 000 daltons and preferably between 50 000 and 200 000 daltons, including 40,000, 60,000, 70,000, 80,000, 90,000, 100,000, 120,000, 140,000, 160,000, 180,000, 220,000, 240,000, 260,000, and 280,000 daltons, and all subranges and values therebetween.

Moreover, these copolymers of ethylene and vinyl acetate that may be used according to the invention preferably have a melting point of greater than 40° C., especially greater than 50° C. and better still greater than or equal to 60° C.

These copolymers may be used alone or as a mixture with at least one compound chosen from tackifying resins as described in the Handbook of Pressure Sensitive Adhesives, edited by Donatas Satas, 3rd edition, 1989, pp. 609-619, waxes as described later, and combinations thereof. The tackifying resins may be chosen especially from rosin, rosin derivatives and hydrocarbon-based resins, and mixtures thereof. Mention may be made in particular of indene-based hydrocarbon-based resins such as resins derived from the polymerization in major proportion of indene monomer and in minor proportion of monomers chosen from styrene, methylindene and methylstyrene, and mixtures thereof. These resins may optionally be hydrogenated. They may have a molecular weight ranging from 290 to 1150. Examples of indene-based resins that may be mentioned in particular include the indene/methyl-styrene/hydrogenated styrene copolymers sold under the name Regalite by the company Eastman Chemical, in particular Regalite R 1100, Regalite R 1090, Regalite R-7100, Regalite R1010 Hydrocarbon Resin and Regalite R1125 Hydrocarbon Resin.

As mixtures based on ethylene/vinyl acetate copolymer, examples that may be mentioned include the products sold under the name Coolbind by the company National Starch.

Preferably, these copolymers are present in the composition in a solids content of greater than or equal to 0.5%, preferably greater than or equal to 0.7% by weight and even better greater than or equal to 0.8% by weight, and preferably greater than or equal to 1% by weight relative to the total weight of the composition.

Preferably, these copolymers are present in the composition in a solids content of between 0.5% and 25% by weight and preferably between 1% and 15% by weight relative to the total weight of the composition.

Examples of mixtures based on ethylene/vinyl acetate copolymers that may be mentioned include the products sold under the name Coolbind by the company National Starch.

Preferably, these copolymers are present in the composition in a solids content of greater than or equal to 0.5%, preferably greater than or equal to 0.7% by weight, better still greater than or equal to 0.8% by weight and preferably greater than or equal to 1% by weight relative to the total weight of the composition.

Preferably, these copolymers are present in the composition in a solids content of between 0.5% and 25% by weight and preferably between 1% and 15% by weight, and more preferably between 1% to 8% by weight relative to the total weight of the composition. This concentration range makes it possible to obtain significant thickening of the fatty phase while at the same time remaining readily manipulable.

These copolymers may be in pure form or may be conveyed in an aqueous phase or an organic solvent phase.

Continuous Oily Phase

The composition according to the invention comprises an oily medium, constituting an oily phase, which forms the continuous phase of the composition. The oily phase of the composition according to the invention is thus a continuous oily phase.

The term “composition with a continuous oily phase” means that the composition has a conductivity, measured at 25° C., of less than 23 μS/cm (microSiemens/cm), the conductivity being measured, for example, using an MPC227 conductimeter from Mettler Toledo and an Inlab 730 conductivity measuring cell. The measuring cell is immersed in the composition so as to remove the air bubbles liable to form between the two electrodes of the cell. The conductivity reading is taken as soon as the conductimeter value has stabilized. A mean of at least three successive measurements is determined.

Preferably, the oily phase of the composition according to the invention is present in an amount at least equal to 30% by weight and preferably in an amount at least equal to 40% by weight, relative to the total weight of the composition.

This continuous oily phase comprises at least one polar wax; it may also comprise at least one oil, and/or at least one lipophilic film-forming polymer.

Oils

The composition according to the invention may comprise one or more oils or non-aqueous fatty substances that are liquid at room temperature (25° C.) and atmospheric pressure (760 mmHg).

The oil may be chosen from volatile oils and/or non-volatile oils, and mixtures thereof.

The oil(s) may be present in the composition according to the invention in a content ranging from 0.1% to 90% by weight and preferably from 0.5% to 60% by weight relative to the total weight of the composition.

For the purposes of the invention, the term “volatile oil” means an oil that is capable of evaporating on contact with the skin or the keratin fibre in less than one hour, at room temperature and atmospheric pressure. The volatile organic solvent(s) and volatile oils of the invention are volatile organic solvents and cosmetic oils that are liquid at room temperature, with a non-zero vapour pressure at room temperature and atmospheric pressure, ranging in particular from 0.13 Pa to 40 000 Pa (10⁻³ to 300 mmHg), in particular ranging from 1.3 Pa to 13 000 Pa (0.01 to 100 mmHg), and more particularly ranging from 1.3 Pa to 1300 Pa (0.01 to 10 mmHg).

The term “non-volatile oil” means an oil that remains on the skin or the keratin fibre at room temperature and atmospheric pressure for at least several hours and that especially has a vapour pressure of less than 10⁻³ mmHg (0.13 Pa).

These oils may be hydrocarbon-based oils, silicone oils or fluoro oils, or mixtures thereof.

The term “hydrocarbon-based oil” means an oil mainly containing hydrogen and carbon atoms and optionally oxygen, nitrogen, sulfur or phosphorus atoms. The volatile hydrocarbon-based oils may be chosen from hydrocarbon-based oils containing from 8 to 16 carbon atoms, and especially branched C₈-C₁₆ alkanes, for instance C₈-C₁₆ isoalkanes of petroleum origin (also known as isoparaffins), for instance isododecane (also known as 2,2,4,4,6-pentamethylheptane), isodecane and isohexadecane, for example the oils sold under the trade names Isopar or Permethyl, branched C₈-C₁₆ esters and isohexyl neopentanoate, and mixtures thereof. Other volatile hydrocarbon-based oils, for instance petroleum distillates, especially those sold under the name Shell Solt by the company Shell, may also be used. The volatile hydrocarbon-based oils may also be chosen from linear C8-C16 alkanes. Examples of linear C8-C16 alkanes that may be mentioned include n-nonadecane (C9), n-decane (C10), n-undecane (C11), n-dodecane (C12), n-tridecane (C13), n-tetradecane (C14), n-pentadecane (C15) and n-hexadecane (C16), and mixtures thereof, and in particular the mixture of n-undecane (C11) and of n-tridecane (C13 sold under the reference Cetiol UT by the company Cognis.

According to one embodiment, a linear volatile alkane that is suitable for use in the invention may be chosen from n-nonadecane, n-undecane, n-dodecane and n-tridecane, and mixtures thereof.

In particular, a linear volatile alkane may be used in the form of an n-undecane/n-tridecane mixture.

Preferably, the n-undecane/n-tridecane weight ratio is in the range from 50/50 to 90/10, preferably from 60/40 to 80/20 and in particular from 65/35 to 75/25.

In particular, a composition of the invention may comprise a mixture of n-undecane and n-tridecane in a 70/30 weight ratio.

The volatile solvent is preferably chosen from volatile hydrocarbon-based oils containing from 8 to 16 carbon atoms, and mixtures thereof.

Volatile oils that may also be used include volatile silicones, for instance volatile linear or cyclic silicone oils, especially those with a viscosity≦8 centistokes (8×10⁻⁶ m²/s) and especially containing from 2 to 7 silicon atoms, these silicones optionally comprising alkyl or alkoxy groups containing from 1 to 10 carbon atoms. As volatile silicone oils that may be used in the invention, mention may be made especially of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Mention may also be made of linear volatile alkyltrisiloxane oils of general formula (I):

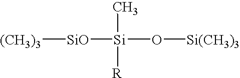

in which R represents an alkyl group containing from 2 to 4 carbon atoms, of which one or more hydrogen atoms may be substituted with a fluorine or chlorine atom.

Among the oils of general formula (I) that may be mentioned are:
3-butyl-1,1,1,3,5,5,5-heptamethyltrisiloxane,
3-propyl-1,1,1,3,5,5,5-heptamethyltrisiloxane, and
3-ethyl-1,1,1,3,5,5,5-heptamethyltrisiloxane,
   corresponding to the oils of formula (I) for which R is, respectively, a butyl group, a propyl group or an ethyl group.

Volatile fluoro solvents such as nonafluoromethoxybutane or perfluoromethylcyclopentane may also be used.

The composition may also comprise at least one non-volatile oil chosen especially from non-volatile hydrocarbon-based oils and/or silicone oils and/or fluoro oils.

Non-volatile hydrocarbon-based oils that may especially be mentioned include:
hydrocarbon-based oils of plant origin, such as triglycerides consisting of fatty acid esters of glycerol, the fatty acids of which may have varied chain lengths from C4 to C24, these chains possibly being linear or branched, and saturated or unsaturated; these oils are especially wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, sesame seed oil, marrow oil, rapeseed oil, blackcurrant oil, evening primrose oil, millet oil, barley oil, quinoa oil, rye oil, safflower oil, candlenut oil, passion flower oil and musk rose oil; or alternatively caprylic/capric acid triglycerides such as those sold by the company Stéarineries Dubois or those sold under the names Miglyol 810, 812 and 818 by the company Dynamit Nobel,
synthetic ethers containing from 10 to 40 carbon atoms;
linear or branched hydrocarbons of mineral or synthetic origin, such as petroleum jelly, polydecenes, hydrogenated polyisobutene such as Parleam, and squalane, and mixtures thereof;
synthetic esters such as oils of formula R1COOR2 in which R1 represents a linear or branched fatty acid residue containing from 1 to 40 carbon atoms and R2 represents an in particular branched hydrocarbon-based chain containing from 1 to 40 carbon atoms, on condition that R1+R2≧10, for instance purcellin oil (cetostearyl octanoate), isopropyl myristate, isopropyl palmitate, C12-C15 alkyl benzoate, hexyl laurate, diisopropyl adipate, isononyl isononanoate, 2-ethylhexyl palmitate, isostearyl isostearate, alkyl or polyalkyl octanoates, decanoates or ricinoleates such as propylene glycol dioctanoate; hydroxylated esters such as isostearyl lactate and diisostearyl malate; and pentaerythritol esters;
fatty alcohols that are liquid at room temperature, containing a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol;
higher fatty acids such as oleic acid, linoleic acid or linolenic acid;
carbonates,
acetates,
citrates,

and mixtures thereof.

The non-volatile silicone oils that may be used in the composition according to the invention may be non-volatile polydimethylsiloxanes (PDMSs), polydimethylsiloxanes comprising alkyl or alkoxy groups, that are pendent and/or at the end of a silicone chain, the groups each containing from 2 to 24 carbon atoms, phenyl silicones, for instance phenyl trimethicones, phenyl dimethicones, phenyltrimethylsiloxydiphenylsiloxanes, diphenyl dimethicones, diphenylmethyldiphenyltrisiloxanes and 2-phenylethyl trimethylsiloxysilicates.

The fluoro oils that may be used in the invention are, in particular, fluorosilicone oils, fluoro polyethers or fluorosilicones, as described in document EP-A-847 752.

Lipophilic Film-Forming Polymers

The composition according to the invention may comprise, besides the copolymer of an alkene and of vinyl acetate, at least lipophilic film-forming polymer.

Among the film-forming polymers that may be used in the composition of the present invention, mention may be made of synthetic polymers, of free-radical type or of polycondensate type, and mixtures thereof.

The lipophilic film-forming polymer may be present in the composition according to the invention in a solids (or active material) content ranging from 0.1% to 30% by weight, preferably from 0.5% to 20% by weight and better still from 1% to 15% by weight relative to the total weight of the composition.

In particular, the mass ratio (by weight of active matter) of the copolymer(s) of an alkene and of vinyl acetate and the lipophilic film-forming polymer(s) will represent from 5 to 0.1, preferably from 3 to 0.2.

In the present invention, the expression “film-forming polymer” means a polymer that is capable, by itself or in the presence of an auxiliary film-forming agent, of forming a macroscopically continuous film that adheres to the keratin fibres.

Free-Radical Film-Forming Polymers

The expression “free-radical film-forming polymer” means a polymer obtained by polymerization of unsaturated and especially ethylenically unsaturated monomers, each monomer being capable of homopolymerizing (unlike polycondensates).

The film-forming polymers of radical-mediated type may be, in particular, vinyl polymers or copolymers, in particular acrylic polymers.

The vinyl film-forming polymers can result from the polymerization of monomers containing ethylenic unsaturation and containing at least one acidic group and/or esters of these acidic monomers and/or amides of these acidic monomers.

Monomers bearing an acid group which may be used are α,β-ethylenic unsaturated carboxylic acids such as acrylic acid, methacrylic acid, crotonic acid, maleic acid or itaconic acid. (Meth)acrylic acid and crotonic acid are preferably used, and more preferably (meth)acrylic acid.

The esters of acidic monomers are advantageously chosen from (meth)acrylic acid esters (also known as (meth)acrylates), especially (meth)acrylates of an alkyl, in particular of a C₁-C₃₀ and preferably C₁-C₂₀ alkyl, (meth)acrylates of an aryl, in particular of a C₆-C₁₀ aryl, and (meth)acrylates of a hydroxyalkyl, in particular of a C₂-C₆ hydroxyalkyl.

Among the alkyl(meth)acrylates that may be mentioned are methyl methacrylate, ethyl methacrylate, butyl methacrylate, isobutyl methacrylate, 2-ethylhexyl methacrylate, lauryl methacrylate and cyclohexyl methacrylate.

Among the hydroxyalkyl(meth)acrylates that may be mentioned are hydroxyethyl acrylate, 2-hydroxypropyl acrylate, hydroxyethyl methacrylate and 2-hydroxypropyl methacrylate.

Among the aryl(meth)acrylates that may be mentioned are benzyl acrylate and phenyl acrylate.

The (meth)acrylic acid esters that are particularly preferred are the alkyl(meth)acrylates.

According to the present invention, the alkyl group of the esters may be either fluorinated or perfluorinated, i.e. some or all of the hydrogen atoms of the alkyl group are substituted with fluorine atoms.

Examples of amides of the acid monomers that may be mentioned are (meth)acrylamides, and especially N-alkyl(meth)acrylamides, in particular of a C₂-C₁₂ alkyl. Among the N-alkyl(meth)acrylamides that may be mentioned are N-ethylacrylamide, N-t-butylacrylamide, N-t-octylacrylamide and N-undecylacrylamide.

The vinyl film-forming polymers may also result from the homopolymerization or copolymerization of monomers chosen from vinyl esters and styrene monomers. In particular, these monomers may be polymerized with acid monomers and/or esters thereof and/or amides thereof, such as those mentioned above.

Examples of vinyl esters that may be mentioned are vinyl acetate, vinyl neodecanoate, vinyl pivalate, vinyl benzoate and vinyl t-butylbenzoate. Styrene monomers that may be mentioned are styrene and α-methylstyrene.

Film-Forming Polycondensates

Among the film-forming polycondensates that may be mentioned are polyurethanes, polyesters, polyesteramides, polyamides, epoxyester resins and polyureas.

The polyurethanes may be chosen from anionic, cationic, nonionic or amphoteric polyurethanes, polyurethane-acrylics, polyurethane-polyvinylpyrrolidones, polyester-polyurethanes, polyether-polyurethanes, polyureas and polyurea-polyurethanes, and mixtures thereof.

The polyesters may be obtained, in a known manner, by polycondensation of dicarboxylic acids with polyols, in particular diols.

The dicarboxylic acid may be aliphatic, alicyclic or aromatic. Examples of such acids that may be mentioned are: oxalic acid, malonic acid, dimethylmalonic acid, succinic acid, glutaric acid, adipic acid, pimelic acid, 2,2-dimethylglutaric acid, azelaic acid, suberic acid, sebacic acid, fumaric acid, maleic acid, itaconic acid, phthalic acid, dodecanedioic acid, 1,3-cyclohexanedicarboxylic acid, 1,4-cyclohexanedicarboxylic acid, isophthalic acid, terephthalic acid, 2,5-norbornanedicarboxylic acid, diglycolic acid, thiodipropionic acid, 2,5-naphthalenedicarboxylic acid or 2,6-naphthalenedicarboxylic acid. These dicarboxylic acid monomers may be used alone or as a combination of at least two dicarboxylic acid monomers. Among these monomers, the ones preferentially chosen are phthalic acid, isophthalic acid and terephthalic acid.

The diol may be chosen from aliphatic, alicyclic and aromatic diols. The diol used is preferably chosen from: ethylene glycol, diethylene glycol, triethylene glycol, 1,3-propanediol, cyclohexanedimethanol and 4-butanediol. Other polyols that may be used are glycerol, pentaerythritol, sorbitol and trimethylolpropane.

The polyesteramides may be obtained in a manner analogous to that of the polyesters, by polycondensation of diacids with diamines or amino alcohols. Diamines that may be used are ethylenediamine, hexamethylenediamine and meta- or para-phenylenediamine. An amino alcohol that may be used is monoethanolamine.

According to an embodiment variant of the composition according to the invention, the lipophilic film-forming polymer may be a polymer dissolved in a liquid fatty phase comprising oils such as those described above (the film-forming polymer is thus said to be a liposoluble polymer). The liquid fatty phase preferably comprises a volatile oil, optionally mixed with a non-volatile oil, the oils possibly being chosen from those mentioned above.

Examples of liposoluble polymers that may be mentioned are copolymers of vinyl ester (the vinyl group being directly linked to the oxygen atom of the ester group and the vinyl ester containing a saturated, linear or branched hydrocarbon-based radical of 1 to 19 carbon atoms, linked to the carbonyl of the ester group) and of at least one other monomer which may be a vinyl ester (other than the vinyl ester already present), an α-olefin (containing from 8 to 28 carbon atoms), an alkyl vinyl ether (in which the alkyl group comprises from 2 to 18 carbon atoms) or an allylic or methallylic ester (containing a saturated, linear or branched hydrocarbon-based radical of 1 to 19 carbon atoms, linked to the carbonyl of the ester group).

These copolymers may be crosslinked with the aid of crosslinking agents, which may be either of the vinyl type or of the allylic or methallylic type, such as tetraallyloxyethane, divinylbenzene, divinyl octanedioate, divinyl dodecanedioate and divinyl octadecanedioate.

Examples of these copolymers that may be mentioned are the following copolymers: vinyl acetate/allyl stearate, vinyl acetate/vinyl laurate, vinyl acetate/vinyl stearate, vinyl acetate/octadecyl vinyl ether, vinyl propionate/allyl laurate, vinyl propionate/vinyl laurate, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethylpropionate/vinyl stearate, allyl dimethylpropionate/vinyl stearate, vinyl propionate/vinyl stearate, crosslinked with 0.2% divinylbenzene, vinyl dimethylpropionate/vinyl laurate, crosslinked with 0.2% divinylbenzene, vinyl acetate/octadecyl vinyl ether, crosslinked with 0.2% tetraallyloxyethane, vinyl acetate/allyl stearate, crosslinked with 0.2% divinylbenzene, vinyl acetate/1-octadecene, crosslinked with 0.2% divinylbenzene, and allyl propionate/allyl stearate, crosslinked with 0.2% divinylbenzene.

According to one particular mode, the composition of the invention comprises, besides the copolymers of an alkene and of vinyl acetate, at least one liposoluble polymer.

In particular, the liposoluble polymer is chosen from copolymers of a vinyl ester (the vinyl group being directly linked to the oxygen atom of the ester group, and the vinyl ester containing a linear or branched saturated hydrocarbon-based radical of 1 to 19 carbon atoms, linked to the carbonyl of the ester group) and of an allylic or methallylic ester (containing a linear or branched saturated hydrocarbon-based radical of 1 to 19 carbon atoms, linked to the carbonyl of the ester group).

Preferably, the liposoluble polymer is chosen from vinyl acetate/allyl stearate copolymers.

Liposoluble film-forming polymers that may also be mentioned include liposoluble copolymers, and in particular those resulting from the copolymerization of vinyl esters containing from 9 to 22 carbon atoms or of alkyl acrylates or methacrylates, and alkyl radicals containing from 10 to 20 carbon atoms.

Such liposoluble copolymers may be chosen from polyvinyl stearate, polyvinyl stearate crosslinked with the aid of divinylbenzene, of diallyl ether or of diallyl phthalate copolymers, polystearyl (meth)acrylate, polyvinyl laurate and polylauryl (meth)acrylate copolymers, it being possible for these poly(meth)acrylates to be crosslinked with the aid of ethylene glycol dimethacrylate or tetraethylene glycol dimethacrylate.

The liposoluble copolymers defined above are known and are described in particular in patent application

FR-A-2 232 303; they may have a weight-average molecular weight ranging from 2000 to 500 000 and preferably from 4000 to 200 000.

According to another mode, at least one copolymer resulting from the copolymerization of vinyl esters containing from 9 to 22 carbon atoms, and in particular polyvinyl laurate copolymers, may be used as additional liposoluble polymer.

According to one particular mode, the composition according to the invention will comprise, besides the copolymers of an alkene and of vinyl acetate, at least one liposoluble polymer chosen from vinyl acetate/allyl stearate copolymers and polyvinyl laurate copolymers, and mixtures thereof.

As liposoluble film-forming polymers that may be used in the invention, mention may also be made of polyalkylenes and in particular copolymers of C2-C20 alkenes, such as polybutene, alkylcelluloses with a linear or branched, saturated or unsaturated C1-C8 alkyl radical, for instance ethylcellulose and propylcellulose, copolymers of vinylpyrrolidone (VP) and in particular copolymers of vinylpyrrolidone and of C2 to C40 and better still C3 to C20 alkene. As examples of VP copolymers which may be used in the invention, mention may be made of the copolymers of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate.

Mention may also be made of silicone resins, which are generally soluble or swellable in silicone oils, which are crosslinked polyorganosiloxane polymers. The nomenclature of silicone resins is known under the name “MDTQ”, the resin being described as a function of the various siloxane monomer units it comprises, each of the letters “MDTQ” characterizing a type of unit.

Examples of commercially available polymethylsilsesquioxane resins that may be mentioned include those sold:

by the company Wacker under the reference Resin MK, such as Belsil PMS MK;

by the company Shin-Etsu under the reference KR-220L.

Siloxysilicate resins that may be mentioned include trimethyl siloxysilicate (TMS) resins such as those sold under the reference SR1000 by the company General Electric or under the reference TMS 803 by the company Wacker. Mention may also be made of the trimethyl siloxysilicate resins sold in a solvent such as cyclomethicone, sold under the name KF-7312J by the company Shin-Etsu, and DC 749 and DC 593 by the company Dow Corning.

Mention may also be made of copolymers of silicone resins such as those mentioned above with polydimethylsiloxanes, for instance the pressure-sensitive adhesive copolymers sold by the company Dow Corning under the reference Bio-PSA and described in document U.S. Pat. No. 5,162,410, or alternatively silicone copolymers derived from the reaction of a silicone resin, such as those described above, and of a diorganosiloxane as described in document WO 2004/073 626.

According to one embodiment of the invention, the film-forming polymer is a film-forming linear block ethylenic polymer, which preferably comprises at least a first block and at least a second block that have different glass transition temperatures (Tg), the first and second blocks being linked together via an intermediate block comprising at least one constituent monomer of the first block and at least one constituent monomer of the second block.

Advantageously, the first and second blocks of the block polymer are mutually incompatible.

Such polymers are described, for example, in documents EP 1 411 069 and WO 04/028 488.

The lipophilic film-forming polymer may also be present in the composition in the form of particles dispersed in a non-aqueous phase. As examples of non-aqueous dispersions of film-forming polymer, mention may be made of acrylic dispersions in isododecane, for instance Mexomer PAP® from the company Chimex, dispersions of particles of a grafted ethylenic polymer, preferably an acrylic polymer, in a liquid fatty phase, the ethylenic polymer advantageously being dispersed in the absence of additional stabilizer at the surface of the particles, as described especially in document WO 04/055 081.

The composition according to the invention may comprise a plasticizer that promotes the formation of a film with the film-forming polymer. Such a plasticizer may be chosen from any compound known to those skilled in the art as being capable of satisfying the desired function.

Polar Waxes

The composition according to the present patent application comprises at least one polar wax.

The term “wax” means a lipophilic compound that is solid at room temperature (25° C.), which may or may not be deformable, with a solid/liquid reversible change of state, having a melting point of greater than or equal to 40° C., which may be up to 120° C. In particular, the waxes that are suitable for use in the invention may have a melting point of greater than or equal to 45° C. and in particular greater than or equal to 55° C.

The term “lipophilic compound” means a compound having an acid number and a hydroxyl number of less than 150 mg KOH/g.

For the purposes of the invention, the melting point corresponds to the temperature of the most endothermic peak observed by thermal analysis (DSC) as described in ISO standard 11357-3; 1999. The melting point of the wax may be measured using a differential scanning calorimeter (DSC), for example the calorimeter sold under the name MDSC 2920 by the company TA Instruments.

The measuring protocol is as follows:

A sample of 5 mg of wax placed in a crucible is subjected to a first temperature rise ranging from −20° C. to 100° C., at a heating rate of 10° C./minute, it is then cooled from 100° C. to −20° C. at a cooling rate of 10° C./minute and is finally subjected to a second temperature increase ranging from −20° C. to 100° C. at a heating rate of 5° C./minute. During the second temperature increase, the variation of the difference in power absorbed by the empty crucible and by the crucible containing the sample of wax is measured as a function of the temperature. The melting point of the compound is the temperature value corresponding to the top of the peak of the curve representing the variation in the difference in absorbed power as a function of the temperature.

The polar waxes may be hydrocarbon-based and/or fluoro waxes, and may be of plant, mineral, animal and/or synthetic origin.

The term “polar wax” means waxes comprising in their chemical structure, in addition to carbon and hydrogen atoms, at least one highly electronegative heteroatom, such as O, N or P.

The polar wax may be present in a content ranging from 1% to 50% by weight, better still from 2% to 40% and even better still from 5% to 30% by weight relative to the total weight of the composition.

Hydrocarbon-based waxes, for instance beeswax, lanolin wax; rice bran wax, carnauba wax, candelilla wax, ouricury wax, Japan wax, berry wax, shellac wax and sumach wax; montan wax, may especially be used as polar wax.

According to one preferred mode, a hydrocarbon-based wax chosen from beeswax, rice bran wax and carnauba wax, and mixtures thereof, will be used.

The waxes obtained by catalytic hydrogenation of animal or plant oils containing linear or branched C8-C32 fatty chains, may also be mentioned.

Among these oils, mention may be made especially of hydrogenated jojoba oil, hydrogenated sunflower oil, hydrogenated castor oil, hydrogenated coconut oil and hydrogenated lanolin oil, bis(1,1,1-trimethylolpropane) tetrastearate sold under the name Hest 2T-4S by the company Heterene, and bis(1,1,1-trimethylolpropane) tetrabehenate sold under the name Hest 2T-4B by the company Heterene.

The wax obtained by hydrogenation of olive oil esterified with stearyl alcohol, sold under the name Phytowax Olive 18 L 57, or the waxes obtained by hydrogenation of castor oil esterified with cetyl alcohol, sold under the name Phytowax Ricin 16L64 and 22L73 by the company Sophim, may also be used. Such waxes are described in patent application FR-A-2 792 190.

Waxes obtained from the reaction of fatty acids with carbohydrates, for instance disaccharides of sucrose type, such as sucrose polybehenate, sold by Croda under the reference Cromaderm B, may also be mentioned.

According to one particular embodiment, the compositions according to the invention may comprise at least one “tacky” wax, i.e. a wax with a tack of greater than or equal to 0.7 N.s and a hardness of less than or equal to 3.5 MPa

Using a tacky wax may especially make it possible to obtain a cosmetic composition that applies easily to the eyelashes, attaches well to the eyelashes and leads to the formation of a smooth, uniform and thickening makeup result.

The tacky wax used may especially have a tack ranging from 0.7 N.s to 30 N.s, in particular greater than or equal to 1 N.s, especially ranging from 1 N.s to 20 N.s, in particular greater than or equal to 2 N.s, especially ranging from 2 N.s to 10 N.s and in particular ranging from 2 N.s to 5 N.s.

The tack of the wax is determined by measuring the change in force (compression force or stretching force) as a function of time, at 20° C., using the texturometer sold under the name TA-TX2i® by the company Rheo, equipped with a conical acrylic polymer spindle forming an angle of 45°.

The measuring protocol is as follows:

The wax is melted at a temperature equal to the melting point of the wax +10° C. The molten wax is poured into a container 25 mm in diameter and 20 mm deep. The wax is recrystallized at room temperature (25° C.) for 24 hours such that the surface of the wax is flat and smooth, and the wax is then stored for at least 1 hour at 20° C. before measuring the tack.

The texturometer spindle is displaced at a speed of 0.5 mm/s then penetrates the wax to a penetration depth of 2 mm. When the spindle has penetrated the wax to a depth of 2 mm, the spindle is held still for 1 second (corresponding to the relaxation time) and is then withdrawn at a speed of 0.5 mm/s.

During the relaxation time, the force (compression force) decreases greatly until it becomes zero, and then, during the withdrawal of the spindle, the force (stretching force) becomes negative and then rises again to the value 0. The tack corresponds to the integral of the curve of the force as a function of time for the part of the curve corresponding to negative values of the force (stretching force). The tack value is expressed in N.s.

The tacky wax that may be used generally has a hardness of less than or equal to 3.5 MPa, in particular ranging from 0.01 MPa to 3.5 MPa, especially ranging from 0.05 MPa to 3 MPa or even ranging from 0.1 MPa to 2.5 MPa.

The hardness is determined by measuring the compression force, which is measured at 20° C. using the texturometer sold under the name TA-XT2 by the company Rheo, equipped with a stainless-steel cylinder 2 mm in diameter, travelling at a measuring speed of 0.1 mm/second, and penetrating into the wax to a penetration depth of 0.3 mm.

The measuring protocol is as follows:

The wax is melted at a temperature equal to the melting point of the wax +10° C. The molten wax is poured into a container 25 mm in diameter and 20 mm deep. The wax is recrystallized at room temperature (25° C.) for 24 hours such that the surface of the wax is flat and smooth, and the wax is then stored for at least 1 hour at 20° C. before measuring the hardness or the tack.

The texturometer spindle is displaced at a speed of 0.1 mm/s then penetrates the wax to a penetration depth of 0.3 mm. When the spindle has penetrated the wax to a depth of 0.3 mm, the spindle is held still for 1 second (corresponding to the relaxation time) and is then withdrawn at a speed of 0.5 mm/s.

The hardness value is the maximum compression force measured divided by the area of the texturometer cylinder in contact with the wax.

Tacky waxes that may be used include a C₂₀-C₄₀ alkyl(hydroxystearyloxy)stearate (the alkyl group containing from 20 to 40 carbon atoms), alone or as a mixture, in particular a C₂₀-C₄₀ alkyl 12-(12′-hydroxystearyloxy)stearate, of formula (II):

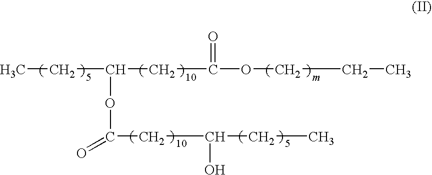

in which m is an integer ranging from 18 to 38, or a mixture of compounds of formula (II).

Such a wax is especially sold under the names Kester Wax K 82 P® and Kester Wax K 80 P® by the company Koster Keunen.

The waxes mentioned above generally have a starting melting point of less than 45° C.

The wax(es) may be present in the form of an aqueous microdispersion of wax. The expression “aqueous microdispersion of wax” means an aqueous dispersion of wax particles in which the size of the wax particles is less than or equal to about 1 μm.

Wax microdispersions are stable dispersions of colloidal wax particles, and are described especially in “Microemulsions Theory and Practice”, L. M. Prince Ed., Academic Press (1977) pages 21-32.

In particular, these wax microdispersions may be obtained by melting the wax in the presence of a surfactant, and optionally of a portion of water, followed by gradual addition of hot water with stirring. The intermediate formation of an emulsion of the water-in-oil type is observed, followed by a phase inversion, with final production of a microemulsion of the oil-in-water type. On cooling, a stable microdispersion of solid wax colloidal particles is obtained.

The wax microdispersions may also be obtained by stirring the mixture of wax, surfactant and water using stirring means such as ultrasound, high-pressure homogenizers or turbomixers.

The particles of the wax microdispersion preferably have mean sizes of less than 1 μm (especially ranging from 0.02 μm to 0.99 μm) and preferably less than 0.5 μm (especially ranging from 0.06 μm to 0.5 μm).

These particles consist essentially of a wax or a mixture of waxes. However, they may comprise a small proportion of oily and/or pasty fatty additives, a surfactant and/or a common liposoluble additive/active agent.

Mention may be made especially of synthetic microwaxes such as the product sold under the name MicroEase 114S® by the company Micro Powders.

Preferably, the polar waxes are chosen from hydrocarbon-based waxes, such as beeswax, lanolin wax; rice bran wax, carnauba wax, candelilla wax, ouricury wax, Japan wax, berry wax, shellac wax and sumach wax; montan wax, hydrogenated jojoba oil, hydrogenated castor oil, hydrogenated lanolin oil, waxes obtained from the reaction of fatty acids with carbohydrates, for instance disaccharides of sucrose type, such as sucrose polybehenate, sold by Croda under the reference Cromaderm B, and tacky waxes, i.e. waxes with a tack of greater than or equal to 0.7 N.s and a hardness of less than or equal to 3.5 MPa, the tacky waxes preferably being hydroxy ester waxes, preferably C20-C40 alkyl(hydroxystearyloxy)stearate waxes such as those sold under the names Kester Wax K 82 P® and Kester Wax K 80 P® by the company Koster Keunen.

More preferably, the polar waxes are chosen from hydrocarbon-based waxes, for instance beeswax, rice bran wax, carnauba wax, hydrogenated jojoba oil, tacky waxes, i.e. waxes with a tack of greater than or equal to 0.7 N.s and a hardness of less than or equal to 3.5 MPa, the tacky waxes preferably being hydroxy ester waxes, preferably C20-C40 alkyl(hydroxystearyloxy)stearate waxes such as those sold under the names Kester Wax K 82 P® and Kester Wax K 80 P® by the company Koster Keunen, and mixtures thereof.

Additional Waxes

The composition according to the invention may also comprise one or more additional waxes different from the polar wax listed above. Such additional waxes are apolar waxes.

The apolar wax under consideration in the context of the present invention satisfies the definition of a wax given above.

The term “apolar” means waxes not comprising in their chemical structure any highly electronegative heteroatoms, such as O, N or P.

The apolar waxes may be hydrocarbon-based or fluoro waxes, and may be of mineral and/or synthetic origin.

The apolar wax may be present in a content ranging from 1% to 50% by weight, better still from 1% to 40% and even better still from 1% to 10% by weight relative to the total weight of the composition.

As illustrations of apolar waxes that are suitable for use in the invention, mention may be made especially of microcrystalline waxes, paraffins and ozokerite; polyethylene waxes, the waxes obtained by Fischer-Tropsch synthesis and waxy copolymers, and also esters thereof; silicone waxes and fluoro waxes.

According to one particular mode, paraffins will be used as additional wax.

The composition may also comprise an aqueous phase.

It may also comprise ingredients commonly used in cosmetics, such as dyestuffs, fillers and fibres, and mixtures thereof.

Aqueous Phase

The composition according to the invention may comprise an aqueous phase in a content of between 0.1% and 20% by weight and preferably between 0.5% and 15% by weight.

The aqueous phase may be made essentially of water; it may also comprise a mixture of water and of water-miscible solvent (miscibility in water of greater than 50% by weight at 25° C.), for instance lower monoalcohols containing from 1 to 5 carbon atoms such as ethanol and isopropanol, glycols containing from 2 to 8 carbon atoms, such as propylene glycol, ethylene glycol, 1,3-butylene glycol and dipropylene glycol, C₃-C₄ ketones and C₂-C₄ aldehydes, and mixtures thereof.

Preferably, the composition according to the invention is anhydrous.

The term “anhydrous” means that the composition comprises water in a content of less than 5% by weight of water, in particular less than 3%, especially less than 2% and more particularly less than 1% by weight of water relative to the total weight of the composition.

More preferentially, the composition of the invention is free of water.

When the composition comprises an aqueous phase, it may comprise at least one hydrophilic film-forming polymer.

The hydrophilic film-forming polymer may be a water-soluble polymer or may be in the form of a dispersion in an aqueous medium.

Examples of water-soluble film-forming polymers that may be mentioned include:
proteins, for instance proteins of plant origin such as wheat or soybean proteins; proteins of animal origin such as keratins, for example keratin hydrolysates and sulfonic keratins;
cellulose polymers such as hydroxyethylcellulose, hydroxypropylcellulose, methylcellulose, ethylhydroxyethylcellulose and carboxymethylcellulose, and also quaternized cellulose derivatives;
acrylic polymers or copolymers, such as polyacrylates or polymethacrylates;
vinyl polymers, for instance polyvinylpyrrolidones, copolymers of methyl vinyl ether and of maleic anhydride, the copolymer of vinyl acetate and of crotonic acid, copolymers of vinylpyrrolidone and of vinyl acetate; copolymers of vinylpyrrolidone and of caprolactam; polyvinyl alcohol;
anionic, cationic, amphoteric or nonionic chitin or chitosan polymers;
gum arabics, guar gum, xanthan derivatives and karaya gum;
alginates and carrageenans;
glycoaminoglycans, and hyaluronic acid and derivatives thereof;
shellac resin, sandarac gum, dammar resins, elemi gums and copal resins;
deoxyribonucleic acid;
mucopolysaccharides such as chondroitin sulfates;

and mixtures thereof.

The film-forming polymer may also be present in the composition in the form of particles dispersed in an aqueous phase, which is generally known as a latex or pseudolatex. The techniques for preparing these dispersions are well known to those skilled in the art.

Aqueous dispersions of film-forming polymer that may be used include the acrylic dispersions sold under the names Neocryl XK-90®, Neocryl A-1070®, Neocryl A-1090®, Neocryl BT-62®, Neocryl A-1079® and Neocryl A-523® by the company Avecia-Neoresins, Dow Latex 432® by the company Dow Chemical, Daitosol 5000 AD® or Daitosol 5000 SJ® by the company Daito Kasey Kogyo; Syntran 5760® by the company Interpolymer, Allianz Opt® by the company Rohm & Haas or the aqueous polyurethane dispersions sold under the names Neorez R-981® and Neorez R-974® by the company Avecia-Neoresins, Avalure UR-405®, Avalure UR-410®, Avalure UR-425®, Avalure UR-450®, Sancure 875®, Avalure UR-445® and Sancure 2060® by the company Noveon, Impranil 85® by the company Bayer, Aquamere H-1511® by the company Hydromer; the sulfopolyesters sold under the brand name Eastman AQ® by the company Eastman Chemical Products, vinyl dispersions, for instance Mexomer PAM®, aqueous polyvinyl acetate dispersions, for instance Vinybran® from the company Nisshin Chemical or those sold by the company Union Carbide, aqueous dispersions of vinylpyrrolidone, dimethylaminopropylmethacrylamide and lauryldimethylpropylmethacrylamidoammonium chloride terpolymer, such as Styleze W from ISP, aqueous dispersions of polyurethane/polyacrylic hybrid polymers such as those sold under the references Hybridur® by the company Air Products or Duromer® from National Starch, and dispersions of core/shell type: for example those sold by the company Atofina under the reference Kynar (core: fluoro; shell: acrylic) or alternatively those described in document U.S. Pat. No. 5,188,899 (core: silica; shell: silicone), and mixtures thereof.

The composition according to the invention may comprise at least one surfactant, chosen especially from amphoteric, anionic, cationic and nonionic surfactants, used alone or as a mixture.

The surfactants may be generally present in the composition in a proportion that may range, for example, from 0.1% to 10% and preferably from 0.5% to 5% by weight.

According to the invention, an emulsifying surfactant appropriately chosen for the production of a water-in-oil emulsion is generally used. In particular, an emulsifying surfactant having at 25° C. an HLB (hydrophilic-lipophilic balance), in the Griffin sense, of less than or equal to 8 may be used.

The HLB value according to Griffin is defined in J. Soc. Cosm. Chem. 1954 (volume 5), pages 249-256.

These surfactants may be chosen from nonionic, anionic, cationic and amphoteric surfactants. Reference may be made to Kirk-Othmer's “Encyclopaedia of Chemical Technology”, volume 22, pp. 333-432, 3rd edition, 1979, published by Wiley, for the definition of the emulsifying properties and functions of surfactants, in particular pp. 347-377 of this reference, for the anionic, amphoteric and nonionic surfactants.

The surfactants preferentially used in the composition according to the invention are chosen from:

a) nonionic surfactants with an HLB of less than 8 at 25° C., optionally combined with one or more nonionic surfactants with an HLB of greater than 8 at 25° C., as mentioned below, for instance:
saccharide esters and ethers such as sucrose stearates, sucrose cocoate, sorbitan stearate and mixtures thereof, for example Arlatone 2121® sold by the company ICI or Span 65V from the company Uniqema;
esters of fatty acids, especially of C₈-C₂₄ and preferably C₁₆-C₂₂, and of a polyol, especially of glycerol or sorbitol, such as glyceryl stearate, sold, for example, under the name Tegin M® by the company Goldschmidt, glyceryl laurate, such as the product sold under the name Imwitor 312® by the company Hüls, polyglyceryl-2 stearate, sorbitan tristearate and glyceryl ricinoleate;
lecithins, such as soybean lecithins (for instance Emulmetik 100 J from Cargill, or Biophilic H from Lucas Meyer);
oxyethylenated and/or oxypropylenated ethers (possibly comprising from 1 to 150 oxyethylene and/or oxypropylene groups) of fatty alcohols (especially of a C₈-C₂₄ and preferably C₁₂-C₁₈ alcohol) such as the oxyethylenated ether of stearyl alcohol containing two oxyethylene units (CTFA name: Steareth-2), such as Brij 72 sold by the company Uniqema;
the cyclomethicone/dimethicone copolyol mixture sold under the name Q2-3225C® by the company Dow Corning;

b) nonionic surfactants with an HLB of greater than or equal to 8 at 25° C., which may be used alone or as a mixture; mention may be made especially of
saccharide esters and ethers such as the mixture of cetylstearyl glucoside and of cetyl and stearyl alcohols, for instance Montanov 68 from SEPPIC;
oxyethylenated and/or oxypropylenated glycerol ethers, possibly comprising from 1 to 150 oxyethylene and/or oxypropylene units;
oxyethylenated and/or oxypropylenated ethers (possibly comprising from 1 to 150 oxyethylene and/or oxypropylene units) of fatty alcohols, especially of C₈-C₂₄ and preferably C₁₂-C₁₈, such as the oxyethylenated ether of stearyl alcohol containing 20 oxyethylene units (CTFA name: Steareth-20), for instance Brij 78 sold by the company Uniqema, the oxyethylenated ether of cetearyl alcohol containing 30 oxyethylene units (CTFA name: Ceteareth-30) and the oxyethylenated ether of the mixture of C₁₂-C₁₅ fatty alcohols comprising 7 oxyethylene units (CTFA name: C₁₂₋₁₅ Pareth-7), for instance the product sold under the name Neodol 25-7® by Shell Chemicals;
fatty acid esters, (especially of C₈-C₂₄ and preferably C₁₆-C₂₂ acid), and of polyethylene glycol (or PEG) (possibly comprising from 1 to 150 oxyethylene units), such as PEG-50 stearate and PEG-40 monostearate sold under the name Myrj 52P® by the company Uniqema;
fatty acid esters (especially of a C₈-C₂₄ and preferably C₁₆-C₂₂ acid) of oxyethylenated and/or oxypropylenated glyceryl ethers (which may comprise from 1 to 150 oxyethylene and/or oxypropylene units), for instance PEG-200 glyceryl monostearate sold under the name Simulsol 220 TM® by the company SEPPIC; glyceryl stearate polyethoxylated with 30 ethylene oxide groups, for instance the product Tagat S® sold by the company Goldschmidt, glyceryl oleate polyethoxylated with 30 ethylene oxide groups, for instance the product Tagat O® sold by the company Goldschmidt, glyceryl cocoate polyethoxylated with 30 ethylene oxide groups, for instance the product Varionic LI 13® sold by the company Sherex, glyceryl isostearate polyethoxylated with 30 ethylene oxide groups, for instance the product Tagat L® sold by the company Goldschmidt, and glyceryl laurate polyethoxylated with 30 ethylene oxide groups, for instance the product Tagat I® from the company Goldschmidt;
fatty acid esters (especially of a C₈-C₂₄ and preferably C₁₆-C₂₂ acid) of oxyethylenated and/or oxypropylenated sorbitol ethers (which may comprise from 1 to 150 oxyethylene and/or oxypropylene units), for instance polysorbate 20 sold under the name Tween 20® by the company Croda, for instance polysorbate 60 sold under the name Tween 60® by the company Uniqema;
dimethicone copolyol, such as the product sold under the name Q2-5220® by the company Dow Corning;
dimethicone copolyol benzoate such as the product sold under the name Finsolv SLB 101® and 201® by the company Finetex;
copolymers of propylene oxide and of ethylene oxide, also known as EO/PO polycondensates;

and mixtures thereof.

The EO/PO polycondensates are more particularly copolymers consisting of polyethylene glycol and polypropylene glycol blocks, for instance polyethylene glycol/polypropylene glycol/polyethylene glycol triblock polycondensates. These triblock polycondensates have, for example, the following chemical structure:

H—(O—CH₂—CH₂)ₐ—(O—CH(CH₃)—CH₂)_{b}—(O—CH₂—CH₂)ₐ—OH,

in which formula a ranges from 2 to 120 and b ranges from 1 to 100.

The EO/PO polycondensates preferably have a weight-average molecular mass ranging from 1000 to 15 000 and better still ranging from 2000 to 13 000.

Advantageously, the EO/PO polycondensates have a cloud point, at 10 g/l in distilled water, of greater than or equal to 20° C. and preferably greater than or equal to 60° C. The cloud point is measured according to ISO standard 1065. As EO/PO polycondensates that may be used according to the invention, mention may be made of the polyethylene glycol/polypropylene glycol/polyethylene glycol triblock polycondensates sold under the name Synperonic®, for instance Synperonic PE/L44® and Synperonic PE/F127®, by the company ICI.

c) anionic surfactants such as:
C₁₆-C₃₀ fatty acid salts, especially amine salts, for instance triethanolamine stearate or 2-amino-2-methyl-1,3-propanediol stearate;
polyoxyethylenated fatty acid salts, especially amine salts or alkali metal salts, and mixtures thereof;
phosphoric esters and salts thereof, such as “DEA oleth-10 phosphate” (Crodafos N 10N from the company Croda) or monocetyl monopotassium phosphate (Amphisol K from Givaudan or Arlatone MAP 160K from the company Uniqema);
sulfosuccinates such as disodium PEG-5 citrate lauryl sulfosuccinate and disodium ricinoleamido MEA sulfosuccinate;
alkyl ether sulfates, such as sodium lauryl ether sulfate;
isethionates;
acylglutamates such as disodium hydrogenated tallow glutamate (Amisoft HS-21 R® sold by the company Ajinomoto), and sodium stearoyl glutamate (Amisoft HS-PF11 sold by the company Ajinomoto), and mixtures thereof;
soybean derivatives, for instance potassium soyate;
citrates, for instance glyceryl stearate citrate (Axol C 62 Pellets from Degussa);
proline derivatives, for instance sodium palmitoyl proline (Sepicalm VG from SEPPIC) or the mixture of sodium palmitoyl sarcosinate, magnesium palmitoyl glutamate, palmitic acid and palmitoyl proline (Sepifeel One from SEPPIC);
lactylates, for instance sodium stearoyl lactylate (Akoline SL from Karlshamns AB);
sarcosinates, for instance sodium palmitoyl sarcosinate (Nikkol sarcosinate PN) or the 75/25 mixture of stearoyl sarcosine and myristoyl sarcosine (Crodasin SM from Croda);
sulfonates, for instance sodium C₁₄-C₁₇ alkyl sec sulfonate (Hostapur SAS 60 from Clariant);
glycinates, for instance sodium cocoyl glycinate (Amilite GCS-12 from Ajinomoto).

Examples of cationic surfactants that may especially be mentioned include:
alkylimidazolidiniums such as isostearylethylimidonium ethosulfate,
ammonium salts such as (C₁₂₋₃₀ alkyl)tri(C₁₋₄ alkyl)ammonium halides, for instance N,N,N-trimethyl-1-docosanaminium chloride (behentrimonium chloride).

The compositions according to the invention may also contain one or more amphoteric surfactants, for instance N-acylamino acids such as N-alkylaminoacetates and disodium cocoamphodiacetate, and amine oxides such as stearamine oxide, or alternatively silicone surfactants, for instance dimethicone copolyol phosphates such as the product sold under the name Pecosil PS 100® by the company Phoenix Chemical.

The composition according to the invention may comprise a water-soluble gelling agent.

The water-soluble gelling agents that may be used in the compositions according to the invention may be chosen from:

homopolymers or copolymers of acrylic or methacrylic acids or the salts and esters thereof, and in particular the products sold under the names Versicol F® or Versicol K® by the company Allied Colloid, Ultrahold 8® by the company Ciba-Geigy, and the polyacrylic acids of Synthalen K type;

copolymers of acrylic acid and of acrylamide sold in the form of the sodium salt thereof under the name Reten® by the company Hercules, sodium polymethacrylate sold under the name Darvan 7® by the company Vanderbilt, and the sodium salts of polyhydroxycarboxylic acids sold under the name Hydagen F® by the company Henkel;

polyacrylic acid/alkyl acrylate copolymers of the Pemulen type;

AMPS (polyacrylamidomethylpropanesulfonic acid partially neutralized with ammonia and highly crosslinked) sold by the company Clariant;

AMPS/acrylamide copolymers of the Sepigel® or Simulgel® type, sold by the company SEPPIC, and

polyoxyethylenated AMPS/alkyl methacrylate copolymers (crosslinked or non-crosslinked),

proteins, for instance proteins of plant origin such as wheat proteins and soybean proteins; proteins of animal origin such as keratins, for example keratin hydrolysates and sulfonic keratins;

polymers of cellulose such as hydroxyethylcellulose, hydroxypropylcellulose, methylcellulose, ethylhydroxyethylcellulose and carboxymethylcellulose, and quaternized cellulose derivatives;

acrylic polymers or copolymers, such as polyacrylates or polymethacrylates;

vinyl polymers, for instance polyvinylpyrrolidones, copolymers of methyl vinyl ether and of malic anhydride, the copolymer of vinyl acetate and of crotonic acid, copolymers of vinylpyrrolidone and of vinyl acetate; copolymers of vinylpyrrolidone and of caprolactam; polyvinyl alcohol;

polymers of natural origin, which are optionally modified, such as:

gum arabics, guar gum, xanthan derivatives, karaya gum;

alginates and carrageenans;

glycosaminoglycans, hyaluronic acid and derivatives thereof;

shellac resin, sandarac gum, dammar resins, elemi gums and copal resins;

deoxyribonucleic acid;

mucopolysaccharides such as chondroitin sulfate,

and mixtures thereof.

Some of these water-soluble gelling agents may also act as film-forming polymers.

The water-soluble gelling polymer may be present in the composition according to the invention in a solids content ranging from 0.01% to 5% by weight, preferably from 0.5% to 2% by weight relative to the total weight of the composition.

Dyestuff

The composition according to the invention may also comprise at least one dyestuff, for instance pulverulent dyes, liposoluble dyes and water-soluble dyes.

The pulverulent dyestuffs may be chosen from pigments and nacres.

The pigments may be white or coloured, mineral and/or organic, and coated or uncoated. Among the mineral pigments that may be mentioned are titanium dioxide, optionally surface-treated, zirconium oxide, zinc oxide or cerium oxide, and also iron oxide or chromium oxide, manganese violet, ultramarine blue, chromium hydrate and ferric blue. Among the organic pigments that may be mentioned are carbon black, pigments of D&C type, and lakes based on cochineal carmine or on barium, strontium, calcium or aluminium.

The nacres may be chosen from white nacreous pigments such as mica coated with titanium or with bismuth oxychloride, coloured nacreous pigments such as titanium mica with iron oxides, titanium mica with, especially, ferric blue or chromium oxide, titanium mica with an organic pigment of the abovementioned type, and also nacreous pigments based on bismuth oxychloride.

The liposoluble dyes are, for example, Sudan Red, D&C Red 17, D&C Green 6, β-carotene, soybean oil, Sudan Brown, D&C Yellow 11, D&C Violet 2, D&C Orange 5, quinoline yellow and annatto.

These dyestuffs may be present in a content ranging from 0.01% to 30% by weight relative to the total weight of the composition.

Fillers

The composition according to the invention may also comprise at least one filler.

The fillers may be chosen from those that are well known to those skilled in the art and commonly used in cosmetic compositions. The fillers may be mineral or organic, and lamellar or spherical. Mention may be made of talc, mica, silica, kaolin, polyamide powders, for instance the Nylon® sold under the trade name Orgasol® by the company Atochem, poly-β-alanine powders and polyethylene powders, powders of tetrafluoroethylene polymers, for instance Teflon®, lauroyllysine, starch, boron nitride, expanded polymeric hollow microspheres such as those of polyvinylidene chloride/acrylonitrile, for instance the products sold under the name Expancel® by the company Nobel Industrie, acrylic powders, such as those sold under the name Polytrap® by the company Dow Corning, polymethyl methacrylate particles and silicone resin microbeads (for example Tospearls® from Toshiba), precipitated calcium carbonate, magnesium carbonate and magnesium hydrogen carbonate, hydroxyapatite, hollow silica microspheres (Silica Beads® from Maprecos), glass or ceramic microcapsules, metal soaps derived from organic carboxylic acids containing from 8 to 22 carbon atoms and in particular from 12 to 18 carbon atoms, for example zinc, magnesium or lithium stearate, zinc laurate and magnesium myristate.

It is also possible to use a compound that is capable of swelling on heating, and especially heat-expandable particles such as non-expanded microspheres of copolymer of vinylidene chloride/acrylonitrile/methyl methacrylate or of acrylonitrile homopolymer copolymer, for instance those sold, respectively, under the references Expancel® 820 DU 40 and Expancel® 007WU by the company Akzo Nobel.

The fillers may represent from 0.1% to 25% and in particular from 1% to 20% by weight relative to the total weight of the composition.

Fibres

The compositions in accordance with the invention may also comprise fibres that allow an improvement in the lengthening effect.

The term “fibre” should be understood as meaning an object of length L and diameter D such that L is very much greater than D, D being the diameter of the circle in which the cross section of the fibre is inscribed. In particular, the ratio L/D (or shape factor) is chosen in the range from 3.5 to 2500, especially from 5 to 500 and in particular from 5 to 150.

The fibres that may be used in the composition of the invention may be mineral or organic fibres of synthetic or natural origin. They may be short or long, individual or organized, for example braided, and hollow or solid. They may have any shape, and may especially have a circular or polygonal (square, hexagonal or octagonal) cross section, depending on the intended specific application. In particular, their ends are blunt and/or polished to prevent injury.

In particular, the fibres have a length ranging from 1 μm to 10 mm, preferably from 0.1 mm to 5 mm and better still from 0.3 mm to 3.5 mm. Their cross section may be within a circle of diameter ranging from 2 nm to 500 μm, preferably ranging from 100 nm to 100 μm and better still from 1 μm to 50 μm. The weight or yarn count of the fibres is often given in denier or decitex, and represents the weight in grams per 9 km of yarn. In particular, the fibres according to the invention may have a yarn count chosen in the range from 0.15 to 30 denier and better still from 0.18 to 18 denier.

The fibres that may be used in the composition of the invention may be chosen from rigid or non-rigid fibres, and may be of synthetic or natural, mineral or organic origin.

Moreover, the fibres may or may not be surface-treated, may be coated or uncoated, and may be coloured or uncoloured.

As fibres that may be used in the composition according to the invention, mention may be made of non-rigid fibres such as polyamide (Nylon®) fibres or rigid fibres such as polyimideamide fibres, for instance those sold under the names Kermel® and Kermel Tech® by the company Rhodia or poly(p-phenyleneterephthalamide) (or aramid) fibres sold especially under the name Kevlar® by the company DuPont de Nemours.

The fibres may be present in the composition according to the invention in a content ranging from 0.01% to 10% by weight, in particular from 0.1% to 5% by weight and more particularly from 0.3% to 3% by weight relative to the total weight of the composition.

Cosmetic Active Agents

As cosmetic active agents that may be used in the compositions in accordance with the invention, mention may be made especially of antioxidants, preserving agents, fragrances, neutralizers, emollients, moisturizers, vitamins and screening agents, in particular sunscreens.

Needless to say, a person skilled in the art will take care to select the optional additional additives and/or the amount thereof such that the advantageous properties of the composition according to the invention are not, or are not substantially, adversely affected by the envisaged addition.

The composition may be in solid, semi-solid or liquid form.

The composition may especially be in the form of a suspension, a dispersion, a solution, a gel, an emulsion, especially a water-in-oil (W/O) emulsion or a multiple emulsion O/W/O), in the form of a cream, a paste, a mousse, a dispersion of vesicles, especially of ionic or nonionic lipids, a two-phase or multiphase lotion, a spray, a powder or a paste, especially a soft paste. Each composition is preferably a leave-in composition.

The composition according to the invention may be manufactured via the known processes generally used in the field of cosmetics.

The composition according to the invention may be conditioned in a container delimiting at least one compartment that comprises the composition, the container being closed by a closing member.

The container is preferably associated with an applicator, especially in the form of a brush comprising an arrangement of bristles maintained by a twisted wire. Such a twisted brush is especially described in patent U.S. Pat. No. 4,887,622. It may also be in the form of a comb comprising a plurality of application members, obtained especially by moulding. Such combs are described, for example, in patent FR 2 796 529. The applicator may be solidly attached to the container, as described, for example, in patent FR 2 761 959. Advantageously, the applicator is solidly attached to a stem, which is itself solidly attached to the closing member.

The closing member may be coupled to the container by screwing. Alternatively, the coupling between the closing member and the container takes place other than by screwing, especially via a bayonet mechanism, by click-fastening or by tightening. The term “click-fastening” in particular means any system involving the passing of a rim or bead of material by elastic deformation of a portion, especially of the closing member, followed by return to the elastically unstressed position of the portion after the rim or bead has been passed.

The container may be at least partly made of thermoplastic material. Examples of thermoplastic materials that may be mentioned include polypropylene and polyethylene.

Alternatively, the container is made of a non-thermoplastic material, especially of glass or metal (or alloy).

The container is preferably equipped with a drainer located in the region of the aperture of the container. Such a drainer makes it possible to wipe the applicator and, optionally, the stem to which it may be solidly attached. Such a drainer is described, for example, in patent FR 2 792 618.

The examples given below are presented as non-limiting illustrations of the invention. Unless otherwise mentioned, the amounts indicated are expressed as mass percentages.

### Example 1

### Mascara

| | Composition | Comparative | Comparative |
|---|---|---|---|
| | 1 | Example A | Example B |
| Ethylene/vinyl acetate | 2.8 | 0.0 | 0.0 |
| copolymer (28% vinyl | | | |
| acetate, Mw = 70 000) | | | |
| Paraffin wax | 2.2 | 2.2 | 2.2 |
| Carnauba wax | 4.7 | 4.7 | 4.7 |
| Beeswax | 8.2 | 8.2 | 8.2 |
| Rice bran wax | 2.2 | 2.2 | 2.2 |
| Vinyl acetate/allyl | 6.7 | 6.7 | 9.5 |
| stearate copolymer | | | |
| (Mexomer PQ from Chimex) | | | |
| Polyvinyl laurate (Mexomer | 0.7 | 0.7 | 0.7 |
| PP from Chimex) | | | |
| Jojoba oil | 1.0 | 1.0 | 1.0 |
| Black iron oxide | 6.4 | 6.4 | 6.4 |
| Talc | 1.0 | 1.0 | 1.0 |
| Bentone | 6.0 | 6.0 | 6.0 |
| Isododecane | qs 100 | qs 100 | qs 100 |

Composition 1 and the Comparative Example A and B are evaluated as regards their application, adhesion and charging, by a panel of 10 experts. Scores ranging from 0 to 10 are attributed for each parameter, a score of 0 being attributed for zero efficiency, and a score of 10 for maximum efficiency.

Composition 1 is judged as being significantly less dry on application, more instantly adherent and more charging than the comparative example A.

In comparison to example B, composition 1 is judged thicker on application, less slippery, and a few more adherent and charging.

### Example 2

### Mascara

| | Composition 2 |
|---|---|
| Ethylene/vinyl acetate copolymer (28% vinyl | 2.8 |
| acetate, Mw = 70 000) | |
| Paraffin wax | 2.2 |
| C20-C40 alkyl (hydroxystearyloxy) stearate | 26.0 |
| (Kester Wax K82P) | |
| Vinyl acetate/allylstearate copolymer | 2.2 |
| (Mexomer PQ from Chimex) | |
| Polyvinyl laurate (Mexomer PP from Chimex) | 0.7 |
| Paraffin wax microbeads (Microease 114S from | 4.4 |
| MicroPowders) | |
| Propylene carbonate | 0.5 |
| Ethanol | 2.0 |
| Black iron oxide | 4.6 |
| Silica | 10.0 |
| Bentone | 1.5 |
| Isododecane | qs 100 |

Composition 1 is pleasant to apply and has no dry sensation. It gives very good immediate charging (from the first brush stroke) and also good final charging.

The above written description of the invention provides a manner and process of making and using it such that any person skilled in this art is enabled to make and use the same, this enablement being provided in particular for the subject matter of the appended claims, which make up a part of the original description.

As used herein, the words “a” and “an” and the like carry the meaning of “one or more.”

The phrases “selected from the group consisting of,” “chosen from,” and the like include mixtures of the specified materials. Terms such as “contain(s)” and the like are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A composition, comprising a continuous oily phase, at least one compound chosen from copolymers of an alkene and of vinyl acetate, and at least one polar wax.

2. The composition according to claim 1, comprising a copolymer of ethylene and of vinyl acetate.

3. A composition according to claim 1, wherein the copolymer(s) of an alkene and of vinyl acetate have a weight-average molecular mass Mw, expressed as polystyrene equivalents, of greater than 30 000 daltons.

4. A composition according to claim 1, wherein the compound chosen from copolymers of an alkene and of vinyl acetate is chosen from copolymers of ethylene and of vinyl acetate comprising between 20% and 40% by weight of vinyl acetate relative to the total weight of the polymer.

5. A composition according to claim 1, wherein the compound chosen from copolymers of an alkene and of vinyl acetate is present in a solids content of greater than or equal to 1% by weight relative to the total weight of the composition.

6. A composition according to claim 1, wherein the compound chosen from copolymers of an alkene and of vinyl acetate is present in a solids content ranging from 0.5% to 25% by weight relative to the total weight of the composition.

7. A composition according to claim 1, further comprising at least one lipophilic film-forming polymer.

8. A composition according to claim 7, wherein the lipophilic film-forming polymer is chosen from copolymers of a vinyl ester and of an allylic or methallylic ester, copolymers resulting from the copolymerization of vinyl esters containing from 9 to 22 carbon atoms or of alkyl acrylates or methacrylates, and alkyl radicals containing from 10 to 20 carbon atoms, and mixtures thereof.

9. A composition according to claim 1, wherein the polar wax is chosen from beeswax, lanolin wax, rice bran wax, carnauba wax, candelilla wax, ouricury wax, Japan wax, berry wax, shellac wax, sumach wax, montan wax, hydrogenated castor oil, hydrogenated lanolin oil, hydrogenated jojoba oil, hydrogenated sunflower oil, hydrogenated coconut oil, bis(1,1,1-trimethylolpropane) tetrastearate, bis(1,1,1-trimethylolpropane) tetrabehenate, the wax obtained by hydrogenation of olive oil esterified with stearyl alcohol, the waxes obtained by hydrogenation of castor oil esterified with cetyl alcohol, the waxes obtained from the reaction of fatty acids with carbohydrates, and waxes with a tack of greater than or equal to 0.7 N.s and a hardness of less than or equal to 3.5 MPa.

10. A composition according to claim 1, wherein the polar wax is chosen from beeswax, lanolin wax, rice bran wax, carnauba wax, candelilla wax, ouricury wax, Japan wax, berry wax, shellac wax, sumach wax, montan wax, hydrogenated castor oil, hydrogenated lanolin oil, hydrogenated jojoba oil, hydrogenated sunflower oil, hydrogenated coconut oil, bis(1,1,1-trimethylolpropane) tetrastearate, bis(1,1,1-trimethylolpropane) tetrabehenate, the wax obtained by hydrogenation of olive oil esterified with stearyl alcohol, the waxes obtained by hydrogenation of castor oil esterified with cetyl alcohol, sucrose polybehenate and the C20-C40 alkyl(hydroxystearyloxy)stearate waxes.

11. A composition according to claim 1, wherein the polar wax is present in an amount of 1% to 50% by weight relative to the total weight of the composition.

12. A composition according to claim 1, wherein the oily phase comprises at least one volatile hydrocarbon-based oil having 8 to 16 carbon atoms.

13. A composition according to claim 1, wherein the oily phase is present in an amount at least equal to 30% by weight relative to the total weight of the composition.

14. A composition according to claim 1, wherein the copolymer(s) of an alkene and of vinyl acetate have a weight-average molecular mass Mw, expressed as polystyrene equivalents, of 60,000-200,000 daltons.

15. A process, comprising applying the composition according to claim 1 to a keratin fibre.

16. A kit comprising:
i) a container delimiting a compartment;
ii) a composition according to claim 1 located inside said compartment.
